(19) United States
(12) Patent Application Publication
(10) Pub. No.: US 2015/0182485 A1
(43) Pub. Date: Jul. 2, 2015

(54) FISH FEED, METHODS FOR PREPARING FEED, AND FEEDING FISH

(71) Applicant: Benemilk Oy, Raisio (FI)

(72) Inventors: Tuula Riitta Susanna AIRAKSINEN, Turku (FI); Knut-Olof LERCHE, Pargas (FI)

(21) Appl. No.: 14/143,244

(22) Filed: Dec. 30, 2013

(51) Int. Cl. A61K 31/202; A61K 35/74; A61K 35/60; A61K 36/02

(52) U.S. Cl. CPC A61K 31/202; A61K 36/02; A61K 35/74; A61K 35/60

(57) ABSTRACT

Fish feed, methods for preparing feed, and methods of providing feed to fish are described herein. A method of providing feed to fish may include providing a first feed composition to the fish prior to the fish growing to a weight of about 50% to about 90% of a desired body weight and providing a second feed composition to the fish after the fish grows to a weight of about 50% to about 90% of the desired body weight. The first feed composition may include a first concentration of at least one of eicosapentaenoic acid and docosahexaenoic acid. The second feed composition may include a second concentration of at least one of eicosapentaenoic acid and docosahexaenoic acid. The second concentration may be at least about 30% greater than the first concentration.

### BACKGROUND

Increasing fish fat levels in farmed fish is of particular interest to fish farmers. Fats in the farmed fish are important for the taste of the fish, the quality of the fish, the size of the fish, and/or the like. It is well known that a fish feed containing a high concentration of fish oil results in the highest quality farmed fish, particularly with respect to taste, quality, and size. However, fish oil is expensive and in short supply, so fish farmers look for alternatives to fish oil.

### SUMMARY

In an embodiment, a method of feeding a fish may include providing a first feed composition to the fish prior to the fish growing to a weight of about 50% to about 90% of a desired body weight and providing a second feed composition to the fish after the fish grows to a weight of about 50% to about 90% of the desired body weight. The first feed composition may include a first concentration of at least one of eicosapentaenoic acid and docosahexaenoic acid. The second feed composition may include a second concentration of at least one of Eicosapentaenoic acid and Docosahexaenoic acid. The second concentration may be at least about 30% greater than the first concentration.

In an embodiment, a method of preparing fish feed may include preparing a first feed composition that includes a first concentration of at least one of Eicosapentaenoic acid and Docosahexaenoic acid. The method may also include preparing a second feed composition that includes a second concentration of at least one of Eicosapentaenoic acid and Docosahexaenoic acid. The second concentration may be at least about 30% or greater than the first concentration. The first feed composition may be configured to be consumed by a fish prior to the fish growing to a weight of about 50% to about 90% of a desired body weight. The second feed composition may be configured to be consumed by the fish after the fish grows to a weight of about 50% to about 90% of the desired body weight.

In an embodiment, a fish feed kit may include a first feed composition having a first concentration of at least one of Eicosapentaenoic acid and Docosahexaenoic acid and a second composition having a second concentration of at least one of Eicosapentaenoic acid and Docosahexaenoic acid. The second concentration may be at least about 30% greater than the first concentration. The fish feed kit may also include a user instruction. The user instruction may be configured to instruct a user to complete at least one of providing the first feed composition prior to the fish growing to a weight of about 50% to about 90% of a desired body weight and providing the second feed composition to the fish after the fish grows to a weight of about 50% to about 90% of the desired body weight.

### BRIEF DESCRIPTION OF THE DRAWINGS

FIG. 1 depicts a flow diagram of an illustrative method of feeding a fish according to an embodiment.

FIG. 2 depicts a flow diagram of an illustrative method of preparing a feed composition according to an embodiment.

FIG. 3 depicts a block diagram of a fish feed kit according to an embodiment.

FIG. 4 is a graphical depiction of a gaping score of a fish provided with feed having a low oil content and a feed having a high oil content according to an embodiment.

FIG. 5 is an illustrative diagram of a filet of fish according to an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The following terms shall have, for the purposes of this application, the respective meanings set forth below.

A “fish” is an animal living wholly in water. The fish may be a vertebrate or an invertebrate. The fish may also be cold-blooded or warm blooded. Some fish may have gills. The fish as described herein are generally farmed fish. Thus, the fish may be raised commercially for food purposes, which is commonly known as aquaculture. The fish can be any genus or species of fish. Illustrative types of fish include, but are not limited to, trout, carp, salmonid, tilapia, sea bass, toothfish, grouper, mullet, tuna, and catfish. Those with ordinary skill in the art will recognize that the term “fish” encompasses other types of fish not specifically described herein.

As used herein, “oil” refers to any hydrophobic or lipophilic compound including, for example, lipids, fats, fatty acids, fatty acid derivatives such as fatty acid esters, triglycerides, and phospholipids.

As used herein, “fish oil” is an oil of marine origin. Particularly, fish oil is derived from tissue of oily fish. In addition, fish oil may be a fatty or an oily extract. Fish oil can be rich in unsaturated fatty acids. Illustrative unsaturated fatty acids include polyunsaturated fatty acids and monounsaturated fatty acids, including monocarboxylic acids having at least one double bond. Unsaturated fatty acids include omega-6 (ω-6) fatty acids such as linoleic acid (LA) and arachidonic acid (AA), omega-3 fatty acids such as eicosapentaenoic acid (EPA) and docosahexaenoic acid (DHA), as well as derivatives and/or precursors thereof. Other unsaturated fatty acids may include myristoleic acid, palmitoleic acid, oleic acid, cis-vaccenic acid, and erucic acid.

As used herein, “vegetable oil” is an oil derived principally from plants. In particular, vegetable oil is obtained from various portions of a plant, such as fruits, seeds and/or nuts of the plant. Illustrative seeds may include oleiferous seeds. Vegetable oil may contain fatty acids and fatty acids esters such as glycerides of fatty acids, such as, for example, oleic acid, palmitic acid, stearic acid, and linoleic acid.

The present disclosure relates generally to a method of feeding a fish. More particularly, the method includes feeding a fish a first feed composition having a first concentration of at least one omega-3 fatty acid so as to build up the initial bulk of the fish until the fish grows to a certain percentage of a desired weight. The method further includes feeding the fish a second feed composition having a higher concentration of omega-3 fatty acid that is at least about 30% higher than the first concentration once the fish grows to the certain percentage of the desired body weight until it is harvested. The fish may generally be harvested before or when the fish grows to the desired body weight. Such a method may result in a fish having a desired taste, quality, and size, as well as a meat having a desired amount of at least one of EPA and/or DHA.

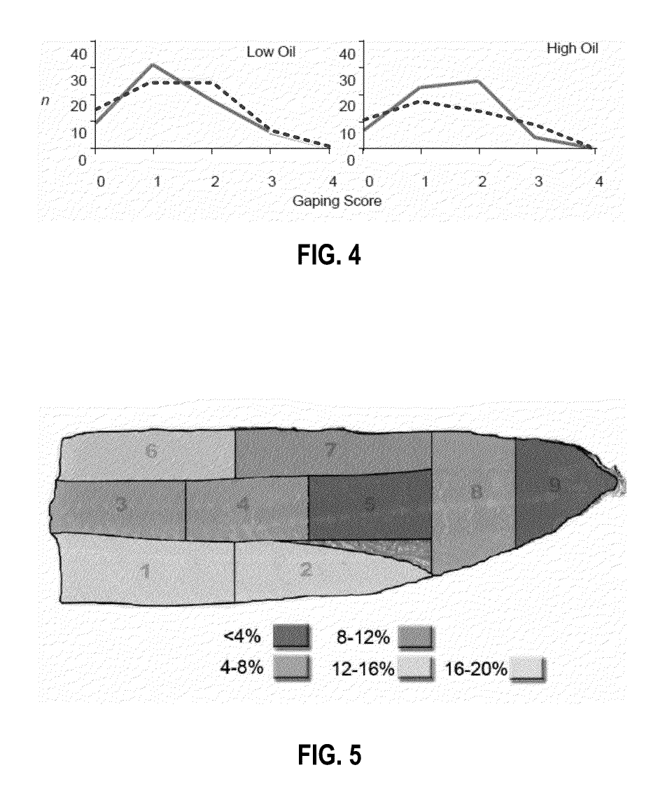

The quality of the fish may include, for example, appearance, texture, eating quality, chemical constituents, food safety, healthiness, and ethical concerns. Such factors affecting quality include, for example, external conditions such as shape, fins, scales, and skin, and internal conditions such as fat content, color, pigment, gaping, texture, bloodspotting, eating quality, and shelf-life. Gaping may refer to holes or gaps in the flesh or meat of the fish. Gaping may be directly correlated to the fish oil content, as shown in FIG. 4. Particularly, fish with a low oil content show more gaping (indicating a lower quality of fish) than fish with a high oil content. The solid lines and the striped lines as shown in FIG. 4 indicate two separate subsets of fish.

In some embodiments, the quality of the fish may include a particular fat content. The fat content may or may not vary with different portions of a filet. FIG. 5 shows a filet chart illustrating a fat content in different portions of a filet. As shown in the chart, portions 1 and 2 of the filet have a fat content of about 16% to about 20% by weight; portions 3, 4, and 8 have a fat content of about 4% to about 8% by weight; portions 5 and 9 have a fat content of less than about 4% by weight; portion 6 has a fat content of about 12% to about 16% by weight; and portion 7 has a fat content of about 8% to about 12% by weight.

In open markets, oils containing at least one of eicosapentaenoic acid and docosahexaenoic acid, such as fish oil and the like, generally costs more than oils that do not contain at least one of eicosapentaenoic acid (EPA) and docosahexaenoic acid (DHA), such as some vegetable oil and the like, because oils that do not contain at least one of eicosapentaenoic acid and docosahexaenoic acid are more abundant than those that do. For example, in some markets, fish oil may cost about 1.5 Euros per kilogram, whereas vegetable oil with no or very low EPA/DHA may cost about 0.6 to about 1 Euro per kilogram. Thus, it may be desirable to use oils lacking eicosapentaenoic acid and docosahexaenoic acid in fish feed compositions in a highest possible concentration to minimize the cost of feed. However, this must be counterbalanced such that the oil does not affect the quality of the fish consuming the feed composition.

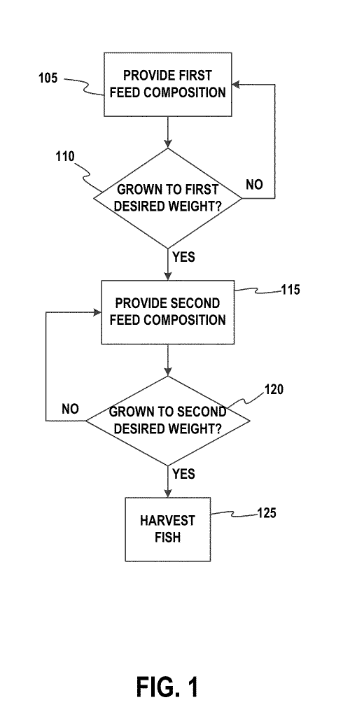

FIG. 1 depicts a flow diagram of a representative method of feeding a fish according to an embodiment. In various embodiments, the method may be performed in a manner such that when a fish consumes the feed composition, the method will maximize the taste, quality, and size of the fish, as described in greater detail herein.

A first feed composition may be provided 105 to the fish such that the fish consumes at least a portion of the first feed composition. The method of providing 105 the first feed composition to the fish is not limited by this disclosure, and may include any method of feeding a fish. Illustrative methods may include sprinkling the first feed composition at a surface of the water in which the fish is located, incorporating the first feed composition into a feeder mechanism, and/or the like.

Providing 105 the first feed composition may also include providing the first feed composition at various feeding intervals. Those with ordinary skill in the art will appreciate appropriate feeding intervals. Illustrative feeding intervals may be about 1 hour to about 96 hours, including but are not limited to, 1 hour, 2 hours, 3 hours, 6 hours, 12 hours, 18 hours, 24 hours, 36 hours, 48 hours, 72 hours, 96 hours, or any value or range between any two of these values (including endpoints). In some embodiments, the feeding interval may be consistent. In other embodiments, the feeding interval may be staggered. In some embodiments, the feeding interval may be dependent upon whether the fish consumes the first feed composition and/or how quickly the fish consumes the first feed composition. In some embodiments, the feeding interval may be dependent upon the type of fish. Thus, for example, a catfish may receive the first feed composition at a different rate than a trout. In some embodiments, the feeding interval may be dependent upon the size of the fish, the size distribution of multiple fish, the age of the fish, the age distribution of multiple fish, the density of fish living in the same habitat, and/or the like.

Providing 105 the first feed composition may include providing an amount of feed. For example, in some embodiments, a fish may receive the feed composition in an amount of about 0.1% to about 3% of the body weight of the fish at each feeding. In some embodiments, a fish may receive the feed composition in an amount of about 0.1% to about 6% of the body weight of the fish at each feeding. In some embodiments, the amount of feed provided 105 may be calculated based upon a desired daily feed ratio. In some embodiments, the amount of feed provided 105 may be dependent on the type of fish, the size or mass/weight of the fish, the size distribution of multiple fish, the age of the fish, the age distribution of multiple fish, the density of fish living in the same habitat, the temperature of the water surrounding the fish, the amount of light that the fish is exposed to, and/or the like.

Providing 105 the first feed composition may include providing the first feed composition in a solid, a semisolid, or a liquid form. Solid forms are not limited by this disclosure and may include, for example, pellets, tablets, capsules, loose particles, flakes, and/or the like. Various ingredients to achieve a solid, semisolid, or liquid form may be added to the first feed composition, as described in greater detail herein.

In various embodiments, the first feed composition may be provided 105 to the fish such that the fish develops a bulk of its mass by consuming the first feed composition. Thus, the first feed composition may be provided 105 prior to the fish growing to a weight of about 50% to about 90% of a desired body weight, about 65% to about 85% of a desired body weight, or about 80% to about 85% of a desired body weight. For example, the first feed composition may be provided 105 prior to the fish growing to a weight of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, or any value or range between any two of these values (including endpoints) of its desired body weight. Thus, a determination 110 may be made as to whether the fish has grown to the percentage of its desired body weight. The determination 110 may generally include weighing the fish. In some embodiments, each fish may be weighed separately. In other embodiments, a plurality of fish may be selected and weighed in aggregate form to determine an average weight of the fish. In other embodiments, the weight of the fish may be estimated based on various factors. For example, the fish may be optically measured to estimate the percentage of desired body weight, or it may be aged to a certain age known or determined to have the percentage of desired body weight. If the fish has not grown to the percentage, the first feed composition may continue to be provided 105. If the fish has grown to the percentage, a second feed composition may be provided 115, as described in greater detail herein.

The desired body weight may generally be selected based on one or more factors. Illustrative factors may be an average full size fish weight, an average harvesting weight, a weight at which the fish possesses a desired taste, a weight at which the fish possesses a desired quality, a weight based on the type of fish, a weight based on the species of fish, a weight based on the age of the fish, and/or the like. In some embodiments, the desired body weight may be determined by measuring a total weight of a sample of fish and using an average weight of the sample as the desired body weight. The desired body weight may be, for example, about 0.1 kg to about 350 kg, about 0.1 kg to about 10 kg, about 0.1 kg to about 14 kg, about 0.1 kg to about 65 kg, about 0.1 kg to about 10 kg, about 0.1 kg to about 100 kg, about 0.1 kg to about 15 kg, about 0.1 kg to about 350 kg, about 0.1 kg to about 8 kg, or about 0.1 kg to about 50 kg. More particularly, the desired body weight may be about, 0.1 kg, about 0.2 kg, about 0.3 kg, about 0.4 kg, about 0.5 kg, about 1 kg, about 2 kg, about 3 kg, about 4 kg, about 5 kg, about 10 kg, about 15 kg, about 20 kg, about 25 kg, about 30 kg, about 35 kg, about 40 kg, about 45 kg, about 50 kg, about 55 kg, about 60 kg, about 65 kg, about 70 kg, about 75 kg, about 80 kg, about 85 kg, about 90 kg, about 95 kg, about 100 kg, about 125 kg, about 150 kg, about 175 kg, about 200 kg, about 250 kg, about 300 kg, about 350 kg, or any value or range between any two of these values (including endpoints).

The first feed composition may generally include a first concentration of at least one of eicosapentaenoic acid and docosahexaenoic acid. In one example, the first concentration refers to the total concentration of eicosapentaenoic acid and docosahexaenoic acid in the first feed composition. As previously described herein, the eicosapentaenoic acid and/or the docosahexaenoic acid may be obtained from any oils containing omega-3 fatty acids, including, for example, various fish oils, certain plant oils, genetically modified plant oils, and marine oils, including algal oils. In some embodiments, the first concentration of eicosapentaenoic acid and/or docosahexaenoic acid may be equal to or less than about 5% by weight of the first feed composition. In particular embodiments, the first concentration may be about 0% by weight to about 5% by weight or about 0% by weight to about 2% by weight of the first feed composition. Other illustrative concentrations may include, but are not limited to, about 0% by weight, about 0.1% by weight, about 0.5% by weight, about 1% by weight, about 1.5% by weight, about 2% by weight, about 2.5% by weight, about 3% by weight, about 3.5% by weight, about 4% by weight, about 4.5% by weight, about 5% by weight, or any value or range between any two of these values (including endpoints). In some embodiments, the first concentration may be selected such that a fish consumes at least a minimum amount of eicosapentaenoic acid and/or docosahexaenoic acid with the first feed composition. The minimum amount of eicosapentaenoic acid and/or docosahexaenoic acid may be, for example, based upon an amount that ensures a desired taste, quality, and/or size of the fish.

In addition to the eicosapentaenoic acid and/or docosahexaenoic acid, the first feed composition may include various other oils. The amount of the various other oils may include about 5% by weight to about 38% by weight of the first feed composition. Illustrative amounts may include, but are not limited to, about 5% by weight, about 10% by weight, about 15% by weight, about 20% by weight, about 25% by weight, about 30% by weight, about 35% by weight, about 38% by weight, or any value or range between any two of these values (including endpoints).

The various other oils are not limited by this disclosure, and may generally include any oils that do not substantially contain eicosapentaenoic acid and/or docosahexaenoic acid, particularly oils suitable for feeding to fish. In some embodiments, the other oils may include, but are not limited to, one or more vegetable oils, nut oils, plant oils, microorganism-derived oils, and animal fats. Illustrative vegetable oils may include rapeseed oil, coconut oil, corn oil, canola oil, cottonseed oil, mustard oil, olive oil, palm oil, peanut oil, safflower oil, sesame oil, soybean oil, sunflower oil, grapefruit seed oil, lemon oil, orange oil, bitter gourd oil, bottle gourd oil, buffalo gourd oil, butternut squash seed oil, egusi seed oil, pumpkin seed oil, watermelon seed oil, linseed oil, truffle oil, olive oil, or a combination thereof. Illustrative nut oils may include, but are not limited to, almond oil, beech nut oil, cashew oil, hazelnut oil, macadamia oil, mongongo nut oil, pecan oil, pine nut oil, pistachio oil, walnut oil, or a combination thereof.

The presence of the various oils in the first feed composition may result in the first feed composition having a fat content. The fat content is generally a percentage of the feed (by weight) that is fat. In some embodiments, the fat content of the first feed composition may be about 5% by weight to about 40% by weight, about 10% by weight to about 35% by weight or about 20% by weight to about 40% by weight. For example, the fat content of the first feed composition may be about 10% by weight, about 15% by weight, about 20% by weight, about 25% by weight, about 30% by weight, about 35% by weight, about 40% by weight, or any value or range between any two of these values (including endpoints).

The fish may be provided 115 with the second feed composition after it grows to a weight of about 50% to about 90% of the desired body weight as described above. In some embodiments, the second feed composition may be provided until the fish reaches the desired body weight. Accordingly, a determination 120 may be made as to whether the fish has reached the desired body weight. The determination 120 may generally include weighing the fish. In some embodiments, each fish may be weighed separately. In other embodiments, a plurality of fish may be selected and weighed in aggregate form to determine an average weight of the fish. In other embodiments, the weight of the fish may be estimated, as described in greater detail herein. If the fish has not reached the desired body weight, the second feed composition may continue to be provided 115 to the fish. If the fish has reached the desired body weight, additional steps may be taken, such as, for example, harvesting 125 the fish. Alternatively, the fish may be optically measured to estimate the percentage of desired body weight, or it may be aged to a certain age known or determined to have the percentage of desired body weight.

Similar to the first feed composition, the second feed composition may generally include a second concentration of at least one of eicosapentaenoic acid and/or docosahexaenoic acid. In one example, the second concentration may be the total concentration by weight of eicosapentaenoic acid and docosahexaenoic acid in the second feed composition. As previously described herein, the eicosapentaenoic acid and/or the docosahexaenoic acid may be obtained from any oils containing omega-3 fatty acids, including, for example, various fish oils, plant oils, genetically modified plant oils, and marine oils, including algal oils. The second concentration may be at least about 30% greater than the first concentration of the first feed composition. In some embodiments, the second concentration may not have an upper limit on the percentage that is greater than the first concentration of the first feed composition. In some embodiments, the second concentration may be about 30% greater than the first concentration to about 150% or more greater than the first concentration. For example, the second concentration may be greater than the first concentration by a factor of about 30%, about 35%, about 40%, about 45%, about 50%, about 55%, about 60%, about 70%, about 75%, about 80%, about 85%, about 90%, about 95%, about 100%, about 105% about 110%, about 115%, about 120%, about 125%, about 130%, about 135%, about 140%, about 145%, about 150%, or greater, as well as any value or range between any two of these values (including endpoints).

In some embodiments, the second concentration may be about 0.1% to about 10% by weight, about 3% to about 8% by weight or about 4% to about 12% by weight of the second feed composition. In particular embodiments, the second concentration may be about 4% by weight to about 8% by weight of the first feed composition. Other illustrative concentrations may include, but are not limited to, about 0.5%, about 1%, about 2%, about 3%, about 4% by weight, about 4.5% by weight, about 5% by weight, about 5.5% by weight, about 6% by weight, about 6.5% by weight, about 7% by weight, about 7.5% by weight, about 8% by weight, about 8.5% by weight, about 9% by weight, about 9.5% by weight, about 10% by weight, about 10.5% by weight, about 11% by weight, about 11.5% by weight, about 12% by weight, or any value or range between any two of these values (including endpoints). In some embodiments, the second concentration may be selected such that a fish consumes at least a minimum amount of eicosapentaenoic acid and/or docosahexaenoic acid with the second feed composition. The minimum amount of eicosapentaenoic acid and/or docosahexaenoic acid may be, for example, based upon an amount that ensures a desired taste, quality, size, and EPA/DHA content of the fish.

In addition to the eicosapentaenoic acid and/or docosahexaenoic acid, the second feed composition may include various other oils. The amount of the various other oils may include about 0.5% by weight to about 16% by weight of the second feed composition. Illustrative amounts may include, but are not limited to, about 0.5% by weight, about 1% by weight, about 2% by weight, about 3% by weight, about 4% by weight, about 5% by weight, about 6% by weight, about 7% by weight, about 8% by weight, about 9% by weight, about 10% by weight, about 11% by weight, about 12% by weight, about 13% by weight, about 14% by weight, about 15% by weight, about 16% by weight, or any value or range between any two of these values (including endpoints).

As with the first feed composition, the various other oils in the second feed composition are not limited by this disclosure, and may generally include any oils that do not substantially contain eicosapentaenoic acid and/or docosahexaenoic acid, particularly oils suitable for feeding to fish. In some embodiments, the other oils may include, but are not limited to, one or more vegetable oils, nut oils, plant oils, microorganism-derived oils, and animal fats. Illustrative vegetable oils may include rapeseed oil, coconut oil, corn oil, canola oil, cottonseed oil, mustard oil, olive oil, palm oil, peanut oil, safflower oil, sesame oil, soybean oil, sunflower oil, grapefruit seed oil, lemon oil, orange oil, bitter gourd oil, bottle gourd oil, buffalo gourd oil, butternut squash seed oil, egusi seed oil, pumpkin seed oil, watermelon seed oil, linseed oil, truffle oil, olive oil, or a combination thereof. Illustrative nut oils may include, but are not limited to, almond oil, beech nut oil, cashew oil, hazelnut oil, macadamia oil, mongongo nut oil, pecan oil, pine nut oil, pistachio oil, walnut oil, or a combination thereof.

As with the first feed composition, the presence of the various oils in the second feed composition may result in the second feed composition having a fat content. The fat content is generally a percentage of the feed (by weight) that is fat. In some embodiments, the fat content of the second feed composition may be about 5% to about 40% by weight, about 10% to about 35% by weight, or 20% by weight to about 40% by weight. For example, the fat content of the second feed composition may be about 10% by weight, 20% by weight, about 25% by weight, about 30% by weight, about 35% by weight, about 40% by weight, or any value or range between any two of these values (including endpoints).

The first feed composition and/or the second feed composition may include at least one additive. The at least one additive may be present in the first feed composition and/or the second feed composition in any amount, particularly in amounts recognized as suitable for fish feed compositions. Illustrative additives may include, but are not limited to, algae, bacteria, metabolic products of bacteria, or a combination thereof.

Oxidative damage (oxidative rancidity) and microbial attack to fish feed compositions and/or components thereof can be a major problem. For example, in the absence of natural antioxidant protection, various fish feed compositions that are rich in polyunsaturated fatty acids are susceptible to oxidative decomposition, which in turn may cause a reduction in the nutritive value of the constituent lipids, proteins, vitamins, and/or the like. Similarly, fish feed compositions with an elevated moisture content are also susceptible to microbial attack and decomposition. Thus, the first feed composition and/or the second feed composition may include at least one preservative. The preservative may generally be a composition that renders the respective feed composition to which it is applied more resistant to oxidative, fungal, and microbial attacks than a similar feed composition not having the preservative applied. Illustrative preservatives may include antioxidants, free-radical scavengers, anti-fungal agents, antimicrobial agents, antibiotics, or a combination thereof. Illustrative preservatives include, but are not limited to, octyl gallate, dodecyl gallate, N-propyl gallate, butylated hydroxyanisole, butylated hydroxytoluene, ethoxyquin, propionic acid, sorbic acid, benzoic acid, acetic acid, formic acid, citric acid, ascorbic acid, gentian violet, potassium bisulfite, potassium metabisulfite, sodium bisulfite, sodium metabisulfite, propylene glycol, a calcium salt, a sodium salt, a potassium salt, or a combination thereof. In some embodiments, the preservative may include an antioxidant. The antioxidant is not limited by this disclosure and may include any antioxidants or combination of antioxidants, particularly those used in fish feed compositions. Illustrative examples of antioxidants may include alpha-carotene, beta-carotene, ethoxyquin, butylated hydroxyanisole (BHA), butylated hydroxytoluene (BHT), cryptoxanthin, lutein, lycopene, zeaxanthin, vitamin A, vitamin C, vitamin E, selenium, alpha-lipoic acid, or a combination thereof.

The first feed composition and/or the second feed composition may include excipients including, for example, carriers, diluents, vehicles, solubilizes, stabilizers, bulking agents, and binders.

In one example, the first feed composition and/or the second feed composition may include at least one carrier material. The at least one carrier material may be present in the first feed composition and/or the second feed composition in any amount, particularly in amounts recognized as suitable for fish feed compositions. The at least one carrier material may provide an amount of carbohydrate or protein to the respective feed composition, may provide bulk to the feed composition, and/or may preserve the quality of the water in which the fish live. In some embodiments, the carrier material may include wheat, barley, potato, pea, broad bean, millet, buckwheat, hyacinth bean, mung bean, sesame, groundnut, safflower, turnips, sugar beet, melon, vegetables, fruits, fiber, corn gluten feed, sunflower hulls, distillers grains, guar hulls, wheat middlings, rice hulls, rice bran, sugar beet pulp, oilseed meals, cottonseed meal, soybean meal, sunflower meal, linseed meal, peanut meal, rapeseed meal, canola meal, dried blood meal, fish by-product, fish meal, dried fish solubles, dried whey, soy protein concentrate, soy flour, yeast, oats, grain sorghums, corn feed meal, rice, rye, corn, barley, aspirated grain fractions, brewers dried grains, corn flour, feeding oat meal, sorghum grain flour, wheat mill run, wheat red dog, hominy feed, wheat flour, wheat bran, wheat germ meal, oat groats, rye middlings, cotyledon fiber, ground grains, wheat grain, corn grain, milo grain, algae, algae meal, microalgae, and the like, or a combination thereof.

In one example, the first feed composition and/or the second feed composition may include at least one binder. The binder may generally provide adhesive properties to the respective feed composition to prevent the feed composition from falling apart in various forms, such as pellet and tablet forms. In some embodiments, the binder may be used to improve the efficiency of a feed manufacturing process, to reduce feed wastage, and/or to produce a water-stable diet. In some embodiments, the binder may make the feed composition easier to digest. The binder may be present in any amount in the respective feed composition, particularly in amounts recognized as acceptable for fish feed compositions. Illustrative amounts of binder may include about 0.5% to about 10% binder by weight of the feed composition, about 0.5% to about 5% by weight of the feed composition, about 0.5% to about 1.5% by weight of the feed composition, and about 1% to about 2% by weight of the feed composition. More specifically, the binder may be about 0.5% by weight, about 1% by weight, about 2% by weight, about 3% by weight, about 4% by weight, about 5% by weight, about 6% by weight, about 7% by weight, about 8% by weight, about 9% by weight, about 10% by weight, or any value or range between any two of these values (including endpoints). Examples of binders may include polysaccharides, proteins, and the like, or a combination thereof. Specific examples of binders may include, but are not limited to, a bentonite, a lignosulfonate, hemicellulose, carboxymethylcellulose, a starchy plant product, an alginate, carrageenan, guar gum, locust bean gum, gum Arabic, agar, high-gluten wheat flour, chitosan, propylene glycol alginate, gelatin, polymethylolcarbamide, a urea-formaldehyde/calcium sulfate mixture, or a combination thereof.

In order for the feed compositions described herein to be successful in providing a benefit to the fish, it must be consumed by the fish. However, feed compositions may be undesirable to fish for various reasons such as appearance (size, shape, and/or color), texture (hard, soft, moist, dry, rough, and/or smooth), density, and attractiveness (smell and/or taste). In some embodiments, the feed composition may be desirable to visual feeding fish and/or chemosensory feeding fish. Thus, the first feed composition and/or the second feed composition may include at least one feeding stimulant. The feeding stimulant may be present in the respective feed composition in any amount, particularly amounts generally recognized as suitable for fish feeds. Illustrative feeding stimulants may include, but are not limited to, squid meal, mussel flesh, shrimp meal, shrimp waste, short-necked clam flesh, marine polychaete worms, blood worms, oligochaete worms, marine fish oils, fish meal, fish solubles, fish protein hydrolysates, soybean protein hydrolysates, L-amino acids, glycine betaine, uridine-5-monophosphate, trimethyl ammonium hydrochloride, or a combination thereof.

Similarly, the first feed composition and/or the second feed composition may include at least one colorant to aid in facilitating ingestion. The colorant may be present in the respective feed composition in any amount, particularly amounts suitable for fish feed compositions. In some embodiments, the colorant may be present in the respective feed composition in a trace amount. In some embodiments, the colorant may be about 0.001% by weight to about 5% by weight of the respective feed composition. More particularly, the colorant may be present in the respective feed composition in an amount of about 0.001% by weight, about 0.01% by weight, about 0.1% by weight, about 0.5% by weight, about 1% by weight, about 2% by weight, about 3% by weight, about 4% by weight, about 5% by weight, or any value or range between any two of these values (including endpoints). Illustrative colorants may include, but are not limited to, capsanthen, lycopene, beta-8′-apo-carotenal, an ethyl ester of beta-8′-apo-carotenoic acid, lutein, cryptoxanthin, violaxanthin, canthaxanthin, xeazanthin, Patent Blue V, curcumin, amaranth, tartrazine, Orange G, Green S, indigo carmine, Brilliant Black, carmoisine, Ponceau 4R, Sunset Yellow FCF, Brown FK, Red 6B, astaxanthin, or a combination thereof.

In various embodiments, the first feed composition and/or the second feed composition may each include at least one amino acid, at least one amino acid derivative, at least one synthetic amino acid, at least one trace element, at least one mineral, at least one vitamin, or any combination thereof. The respective feed composition may include various portions generally included in particular amounts that are sufficient to provide beneficial nutritional and dietary needs of the fish that is to consume the feed compositions. For example, the first feed composition may include a carbohydrate portion and a vitamin portion, each in an amount sufficient to provide beneficial nutritional and dietary needs of the fish.

The amino acid may be an essential amino acid, including any combination of leucine, lysine, histidine, valine, arginine, threonine, isoleucine, phenylalanine, methionine, tryptophan, any derivative thereof, or any combination thereof. In some embodiments, the amino acid may be a non-essential amino acid, including any combination of alanine, asparagine, aspartate, cysteine, glutamate, glutamine, glycine, proline, serine, tyrosine, any derivative thereof, or any combination thereof. The amino acid and/or any derivative thereof may also include amino acids or derivatives of either non-essential or essential amino acids. The amino acid may generally be included in the respective feed composition to provide a nutritional aid in various physiological processes in the fish, such as, for example, increasing muscle mass, providing energy, aiding in recovery, and/or the like. In some embodiments, the amino acid may be obtained from a premix composition. In some embodiments, the amino acid may be a synthetic amino acid. A synthetic amino acid may generally be an amino acid that is chemically synthesized and is not one of the 20 amino acids naturally occurring in nature.

The mineral may be any mineral that is a generally recognized as safe (GRAS) mineral or a combination of such minerals. The mineral may further be obtained from any mineral source that provides a bioavailable mineral. In some embodiments, the mineral may be a mineral ion selected from at least one of calcium, sodium, magnesium, potassium, phosphorous, zinc, selenium, manganese, iron, cobalt, copper, iodine, molybdenum, and/or the like. In some embodiments, the mineral may be selected from one or more of a sodium salt, a calcium salt, a magnesium salt, a cobalt salt, a manganese salt, a potassium salt, an iron salt, and a zinc salt. In some embodiments, the mineral may be selected from copper sulfate, copper oxide, selenium yeast, a chelated mineral, and/or the like. Illustrative examples of sodium salts include monosodium phosphate, sodium acetate, sodium chloride, sodium bicarbonate, disodium phosphate, sodium iodate, sodium iodide, sodium tripolyphosphate, sodium sulfate, sodium selenite, and/or the like. Illustrative examples of calcium salts include calcium acetate, calcium carbonate, calcium chloride, calcium gluconate, calcium hydroxide, calcium iodate, calcium iodobehenate, calcium oxide, anhydrous calcium sulfate, calcium sulfate dehydrate, dicalcium phosphate, monocalcium phosphate, tricalcium phosphate, and/or the like. Illustrative magnesium salts include magnesium acetate, magnesium carbonate, magnesium oxide, magnesium sulfate, and/or the like. Illustrative cobalt salts include cobalt acetate, cobalt carbonate, cobalt chloride, cobalt oxide, cobalt sulfate, and/or the like. Illustrative examples of manganese salts include manganese carbonate, manganese chloride, manganese citrate, manganese gluconate, manganese orthophosphate, manganese oxide, manganese phosphate, manganese sulfate, and/or the like. Illustrative examples of potassium salts include potassium acetate, potassium bicarbonate, potassium carbonate, potassium chloride, potassium iodate, potassium iodide, potassium sulfate, and/or the like. Illustrative examples of iron salts include iron ammonium citrate, iron carbonate, iron chloride, iron gluconate, iron oxide, iron phosphate, iron pyrophosphate, iron sulfate, reduced iron, and/or the like. Illustrative examples of zinc salts include zinc acetate, zinc carbonate, zinc chloride, zinc oxide, zinc sulfate, and/or the like.

The vitamin may include any combination of vitamins including, without limitation, vitamin A, vitamin B, vitamin C, vitamin D, vitamin E, vitamin K, and/or the like. Specific examples of vitamin B include thiamine (vitamin B1), riboflavin (vitamin B2), niacin (vitamin B3), pantothenic acid (vitamin B5), pyridoxine (vitamin B6), biotin (vitamin B7), folic acid (vitamin B9), cobalamin (vitamin B12), choline (vitamin Bₚ), or a combination thereof.

In various embodiments, the first feed composition and/or the second feed composition may include a micronutrient mixture. Micronutrient mixtures are not limited by this disclosure and may generally contain any micronutrient mixture now known or later developed. The micronutrient mixture may include various components, such as at least one vitamin and at least one mineral, as described in greater detail herein. In some embodiments, the micronutrient mixture may be present in a premix composition.

The feeding methods and fish feed compositions described herein may be applicable to a variety of fishes, including, but not limited to, a trout, a carp, a salmon, a tilapia fish, a sea bass, a toothfish, a grouper, a mullet, a tuna fish, or a catfish. The desired body weight may vary with different types of fishes. For example, where the fish is a trout, the desired body weight may be about 0.1 kg to about 10 kg. Where the fish is a carp, the desired body weight may be about 0.1 kg to about 14 kg. Where the fish is a salmonid, the desired body weight may be about 0.1 kg to about 65 kg. Where the fish is a tilapia fish, the desired body weight may be about 0.1 kg to about 10 kg. Where the fish is a sea bass, the desired body weight may be about 0.1 kg to about 100 kg. Where the fish is a toothfish, the desired body weight may be about 0.1 kg to about 15 kg. Where the fish is a grouper, the desired body weight may be about 0.1 kg to about 350 kg. Where the fish is a mullet, the desired body weight may be about 0.1 kg to about 8 kg. Where the fish is a catfish, the desired body weight may be about 0.1 kg to about 50 kg.

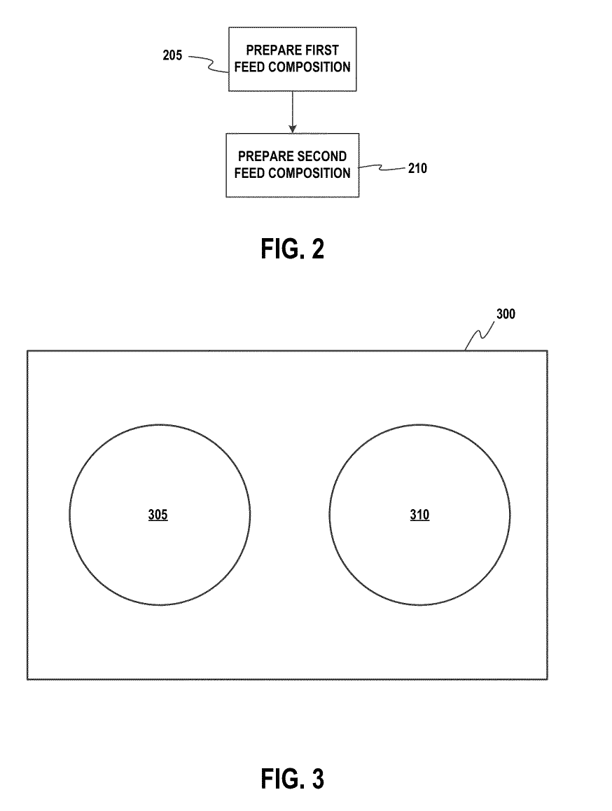

FIG. 2 depicts a flow diagram of a method of preparing a fish feed according to an embodiment. The method may generally include preparing 205 the first feed composition as described herein and preparing 210 the second feed composition as described herein. Preparation 205, 210 is not limited by this disclosure and may include any number of processes, such as, for example, combining, mixing, processing, and/or the like. Processing may include forming the first feed composition and/or the second feed composition into a capsule, a shell, a pellet, a tablet, a granular material, a flake, and/or the like. Accordingly, processing may include pressing, molding, extruding, grinding, pelletizing, encapsulating, granulating, and/or the like. Pressing may include, for example, applying a pressure to an amount of the respective feed composition. Molding may include, for example, open molding, compression molding, injection molding, centrifugal molding, or the like. Extruding may include, for example, forming an amount of the respective feed composition by forcing the feed composition through a die having a desired shape and size.

Grinding may be performed by various grinding devices known to those having ordinary skill in the art, such as a hammer mill, a roller mill, a disk mill, or the like. The respective feed composition and/or portions thereof may be ground to various sizes, such as particle sizes (for instance, measured in millimeters), mesh sizes, surface areas, or the like. According to some embodiments, the respective feed composition and/or portions thereof may be ground to an average particle size of about 0.05 mm to about 10 mm. More particularly, the respective feed composition may be ground to produce a granular material having an average particle size of about 0.05 mm, about 0.1 mm, about 0.2 mm, about 0.5 mm, about 1.0 mm, about 2.0 mm, about 3.0 mm, about 4.0 mm, about 5.0 mm, about 6.0 mm, about 7.0 mm, about 8.0 mm, about 9.0 mm, about 10.0 mm, or any value or range between any two of these values. In some embodiments, the respective composition may be ground so that about 20% to 50% of the ground feed composition is retained by a mesh having openings with a size of about 10 mm and so that about 70% to about 90% of the ground feed composition is retained by a mesh having openings with a size of about 1 mm. In some embodiments, the respective feed compositions and/or various portions thereof may have a varying distribution of particle sizes based upon the ingredients.

Grinding may provide various benefits, such as improving certain characteristics of the respective feed composition. For instance, even and fine particle size may improve the mixing of different ingredients. According to certain embodiments, grinding may be configured to decrease a particle size of certain components of the respective feed composition, for example, to increase the surface area open for enzymes in the intestinal tract, which may improve the digestibility of nutrients, and/or to increase the palatability of the feed.

In some embodiments, the granular material or powder may be used in subsequent processes such as molding, extrusion, and/or tableting. In some embodiments, processing may include drying the respective feed composition. Drying may generally be completed to remove any excess water or other undesired materials, as well as to provide a material that is suitable for encapsulation, pelletizing, extrusion, grinding, pressing and/or the like.

In some embodiments, preparing 205, 210 the fish feed may be performed as part of the preparation of a fish feed kit, such as the kit 300 shown in FIG. 3. The kit 300 may generally have a first container 305 and a second container 310. The first container 305 may contain the first feed composition, as described in greater detail herein. The second container 310 may contain the second feed composition, as described in greater detail herein. The contents of the first container 305 may be provided to the fish prior to the fish growing to the percentage of the desired body weight, as described in greater detail herein. Thereafter, the contents of the second container 310 may be provided to the fish after the fish grows to the percentage of the desired body weight. In some embodiments, the kit may include one or more user instructions for providing the first feed composition and the second feed composition to the fish as described herein. In some embodiments, the one or more user instructions may be on the first container and/or the second container. In some embodiments, the user instruction may be printed on a sheet or in a brochure.

### EXAMPLES

### Example 1

### Preparing Fish Feed

A solid fish feed will be prepared at a manufacturing facility. The solid fish feed will be manufactured for feeding to farmed sockeye salmon to increase the quality and taste of the salmon when harvested and consumed by humans. The solid fish feed will also be manufactured to produce sockeye salmon that are healthy for human consumption and contain a desirable amount of EPA and DHA. The solid fish feed will have a first feed composition and a second feed composition.

The first feed composition includes a fat content of 20% by weight, of which 2% by weight of first feed composition is eicosapentaenoic acid and docosahexaenoic acid and 18% by weight of the first feed composition is a 50:50 mixture of rapeseed oil and peanut oil. The remaining 80% by weight of the first feed composition includes a mixture of algae meal, wheat, citric acid, ascorbic acid, guar gum, soybean protein hydrolysates, capsanthen for color, leucine, lysine, histidine, tryptophan, monosodium phosphate, calcium carbonate, vitamin A, vitamin K, thiamine, biotin, and folic acid. The first feed composition will be pressed and pelletized to form a pellet material that is desirable to the salmon.

The second feed composition will include a fat content of 35% by weight, of which 10% by weight of the second feed composition is eicosapentaenoic acid and docosahexaenoic acid, and 25% by weight of the second feed composition is the 50:50 mixture of rapeseed oil and peanut oil. The remaining 65% by weight of the second feed composition includes a mixture of algae meal, wheat, citric acid, ascorbic acid, guar gum, soybean protein hydrolysates, capsanthen for color, leucine, lysine, histidine, tryptophan, monosodium phosphate, calcium carbonate, vitamin A, vitamin K, thiamine, biotin, and folic acid. Similar to the first feed composition, the second feed composition will also be pressed and pelletized to form a pellet material that is desirable to the salmon.

### Example 2

### Fish Feed Kit

A kit will be prepared for shipping to farmers of sockeye salmon. The kit will contain a first container and a second container. The first container will have 100 kg of the first feed composition described above with respect to Example 1. The first container will include instructions for the farmer for providing the first feed composition until the salmon grows to 85% of a desired body weight. The second container will have 100 kg of the second feed composition described above with respect to Example 1. The second container will include instructions for the farmer for providing the second feed composition after the salmon has grown to 85% of the desired body weight. The instructions will also direct the farmer to continue feeding the fish the second feed composition until the salmon reaches the desired body weight.

### Example 3

### Providing Feed to Fish

A farmer will receive the kit as described with respect to Example 2. To test for efficacy, the farmer will provide the feed compositions described above with respect to Example 1 to a first tank containing a test group of 1000 fingerling sockeye salmon and a standard fish feed composition to a second tank containing a control group of 1000 fingerling sockeye salmon. The farmer will select a desired weight of the sockeye salmon to be about 7.0 kg, which is a desired weight for harvesting.

The first tank containing the test group of salmon will initially be provided with the first feed composition. Feeding the salmon will be completed on a twice-daily basis such that the salmon are able to consume the first feed composition every 12 hours. The salmon will be weighed in aggregate form every three weeks to determine the average weight of the test salmon. When the average weight of the salmon reaches about 5.95 kg (85% of the desired 7.0 kg weight), the farmer will cease feeding the first food composition to the salmon. Instead, the farmer will provide the second feed composition to the salmon. The rate and method of feeding and weighing will remain the same with the second feed composition. Once the average weight of the salmon reaches about 7 kg, the farmer will harvest the salmon.

The second tank containing the control group of salmon will be provided with a standard feed composition via the same methods and intervals described above with respect to the test group of salmon. In addition, the control group of salmon will be weighed in aggregate form at substantially the same time as the test group of salmon to determine an average control salmon weight. Once the average weight of the salmon reaches about 7 kg, the farmer will harvest the salmon.

Upon harvesting, the farmer will note that the cost of feeding the test group of salmon will be significantly less than the cost of feeding the control group of salmon because of the decreased concentration of fish oil in the first feed composition. The farmer will also note that the test group of salmon will be of equal or higher quality with a pleasing taste. Thus, the farmer will sell the test group of salmon more quickly and at a higher price than the control group because it will be in higher demand. As the cost of feeding the test group of salmon is significantly less than the cost of feeding the control group of salmon, the farmer will realize a larger profit margin by selling the test group of salmon, even if the farmer sells both the test group and the control group of salmon at the same price.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera As a non-limiting example, each range discussed herein can be readily broken down into a lower third, middle third and upper third, et cetera As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method of feeding a fish, the method comprising:
providing a first feed composition to the fish prior to the fish growing to a weight of about 50% to about 90% of a desired body weight, wherein the first feed composition comprises a first concentration of at least one of eicosapentaenoic acid and docosahexaenoic acid; and
providing a second feed composition to the fish after the fish grows to a weight of about 50% to about 90% of the desired body weight, wherein the second feed composition comprises a second concentration of at least one of eicosapentaenoic acid and docosahexaenoic acid,
wherein the second concentration is at least about 30% greater than the first concentration.

2. The method of claim 1, wherein providing the first feed composition comprises providing the first feed composition to the fish prior to the fish growing to a weight of about 65% to about 85% of the desired body weight.

3. The method of claim 1, wherein the first concentration is about 0% by weight to about 5% by weight of the first feed composition.

4. The method of claim 1, wherein the second concentration is about 0.1% by weight to about 12% by weight of the second feed composition.

5. The method of claim 1, wherein the first feed composition comprises fish oil, marine oil, oils derived from genetically modified plant sources, or a combination thereof.

6. The method of claim 1, wherein the first feed composition comprises a fat content of about 5% by weight to about 35% by weight.

7. The method of claim 1, wherein the first feed composition comprises at least one of a vegetable oil, a nut oil, a plant oil, a microorganism-derived oil, an animal fat, or a combination thereof.

8. The method of claim 1, wherein the first feed composition further comprises at least one additive comprising an alga, a bacterium, a metabolic product of a bacterium, or a combination thereof.

9. The method of claim 1, wherein the first feed composition further comprises at least one feeding stimulant comprising squid meal, mussel flesh, shrimp meal, shrimp waste, short-necked clam flesh, marine polychaete worms, blood worms, oligochaete worms, marine fish oils, fish meal, fish solubles, fish protein hydrolysates, soybean protein hydrolysates, L-amino acids, glycine betaine, uridine-5-monophosphate, trimethyl ammonium hydrochloride, or a combination thereof.

10. The method of claim 1, wherein the second feed composition comprises fish oil, marine oil, oils derived from genetically modified plant sources, or a combination thereof.

11. The method of claim 1, wherein the second feed composition comprises at least one of a vegetable oil, a nut oil, a plant oil, a microorganism-derived oil, an animal fat, or a combination thereof.

12. The method of claim 1, wherein the second feed composition comprises a fat content of about 5% by weight to about 35% by weight.

13. The method of claim 1, wherein the second feed composition further comprises at least one additive comprising an alga, a bacterium, a metabolic product of a bacterium, or a combination thereof.

14. The method of claim 1, wherein the second feed composition further comprises at least one feeding stimulant comprising squid meal, mussel flesh, shrimp meal, shrimp waste, short-necked clam flesh, marine polychaete worms, blood worms, oligochaete worms, marine fish oils, fish meal, fish solubles, fish protein hydrolysates, soybean protein hydrolysates, L-amino acids, glycine betaine, uridine-5-monophosphate, trimethyl ammonium hydrochloride, or a combination thereof.

15. The method of claim 1, wherein the fish is a trout, a carp, a salmonid, a tilapia fish, a sea bass, a toothfish, a grouper, a mullet, a tuna fish, or a catfish.

16. The method of claim 1, wherein the desired body weight is about 0.1 kg to about 350 kg.

17. A method of preparing a fish feed, the method comprising:
preparing a first feed composition having a first concentration of at least one of eicosapentaenoic acid and docosahexaenoic acid; and
preparing a second feed composition having a second concentration of at least one of eicosapentaenoic acid and docosahexaenoic acid,
wherein the second concentration is at least about 30% greater than the first concentration,
wherein the first feed composition is configured to be consumed by a fish prior to the fish growing to a weight of about 50% to about 90% of a desired body weight, and
wherein the second feed composition is configured to be consumed by the fish after the fish grows to a weight of about 50% to about 90% of the desired body weight.

18. The method of claim 17, wherein the first feed composition is configured to be consumed by the fish prior to the fish growing to a weight of about 65% to about 85% of the desired body weight.

19. The method of claim 17, wherein the first concentration is about 0% by weight to about 5% by weight of the first feed composition.

20. The method of claim 17, wherein the second concentration is about 4% by weight to about 12% by weight of the second feed composition.

21. The method of claim 17, wherein the first feed composition comprises fish oil, marine oil, oils derived from genetically modified plant sources, or a combination thereof.

22. The method of claim 17, wherein the first feed composition comprises a fat content of about 10% to about 30% by weight.

23. The method of claim 17, wherein the first feed composition comprises at least one of a vegetable oil, a nut oil, a plant oil, a microorganism-derived oil, an animal fat, or a combination thereof.

24. The method of claim 17, wherein the second feed composition comprises fish oil, marine oil, oils derived from genetically modified plant sources, or a combination thereof.

25. The method of claim 17, wherein the second feed composition comprises at least one of a vegetable oil, a nut oil, a plant oil, a microorganism-derived oil, an animal fat, or a combination thereof.

26. The method of claim 17, wherein the second feed composition comprises a fat content of about 5% by weight to about 35% by weight.

27. A fish feed kit comprising:
a first feed composition having a first concentration of at least one of eicosapentaenoic acid and docosahexaenoic acid;
a second feed composition having a second concentration of at least one of eicosapentaenoic acid and docosahexaenoic acid; and
an user instruction,
wherein the second concentration is at least about 30% greater than the first concentration, and
wherein the user instruction is configured to instruct a user to feed the first feed composition to a fish prior to the fish growing to a weight of about 50% to about 90% of a desired body weight, feed the second feed composition to the fish after the fish grows to a weight of about 50% to about 90% of the desired body weight, or both.
